(19) United States
(12) Patent Application Publication
(10) Pub. No.: US 2019/0083366 A1
(43) Pub. Date: Mar. 21, 2019

(54) MOUTHWASH GRANULES FOR AN AQUEOUS SOLUTION

(71) Applicant: MARLINSPIKE GROUP, INC., Solana Beach, CA (US)

(72) Inventors: Pratik SHAH, Solana Beach, CA (US); John Christopher CARTER, Keswick (CA)

(21) Appl. No.: 15/709,029

(22) Filed: Sep. 19, 2017

(51) Int. Cl. A61K 8/02; A61Q 11/00; A61K 8/49; A61K 8/19; A61K 8/365

(52) U.S. Cl. CPC A61K 8/0225; A61Q 11/00; A61K 8/4926; A61K 2800/222; A61K 8/365; A61K 2800/92; A61K 8/19

(57) ABSTRACT

The present invention relates to a dry mouthwash comprising granules that reconstitute in an aqueous solution, such as water, to produce a solution that tastes and functions as a typical liquid mouthwash.

### FIELD OF THE INVENTION

The present invention relates to dry mouthwash granules that reconstitute in an aqueous solution, such as water, to produce a solution that tastes and functions as a typical liquid mouthwash.

### BACKGROUND

The current mouthwash industry and consumers of mouthwash currently face several challenges. People usually don't carry mouthwash to use post meal or drink and generally rely on a mint or gum of some kind to provide fresh breath when not at home. However, these are often filled with sugar and are not beneficial for oral hygiene and maintenance. Therefore, a product has been designed to provide a convenient and effective mouthwash for travel and use outside the home.

Liquid mouthwash is difficult for consumers to carry with them, especially when consumers desire to use mouthwash when they are traveling. Traveling with liquid mouthwash is difficult as containers of liquid mouthwash in the necessary quantity of volume can be bulky and inconvenient in most circumstances. Liquid are often prohibited to carry on flights when traveling.

Tablets generally take a long time to dissolve, particularly in cold water or other aqueous environments. However, powders tend to float on the surface of aqueous environments and may not be useful for us in a reconstituted mouthwash. Accordingly, there is a need for a dry mouthwash granule that will quickly dissolve in an aqueous medium and produce a mouthwash.

### BRIEF DESCRIPTION OF THE DRAWINGS

FIG. 1 is a flow chart for the general manufacturing procedure.

### DETAILED DESCRIPTION

### Definitions

The following are definitions for the present disclosure.

The term “active components”, as used herein, include antiseptic, remineralising agents, anti-caries agents, anticalculus agents, moisturising agents, breath freshening agents and desensitising agents. Active components are preferably antiseptic such as cetylpyridinium chloride (CPC) or a hydrate thereof preferably Monohydrate, chlorhexidine, zinc salts, triclosan and/or the like. Most preferred is cetylpyridinium chloride.

The term “desensitizing agent”, as used herein, refers to compounds that can reduce pain or discomfort, including, but not limited to, oral pain or discomfort. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

An effervescent substance is a substance that, upon dissolution in an aqueous medium, produces gas.

An effervescent reaction is the production of gas in an aqueous medium upon dissolution of a substance.

The term “carbonate salt” as used herein, includes salts that comprise one or more anions selected from: carbonate (CO₃²⁻) and bicarbonate (HCO₃⁻).

### Formulation Components and Function

The following is a non-limiting list of ingredients that can be used with the present invention.

| Ingredient | Function |
|---|---|
| Cetylpyridinium Chloride | Monograph Active Components |
| Monohydrate | |
| Menthol | Flavor enhancement |
| Sodium Bicarbonate #2 | Effervescent Disintegration System |
| Tartaric Acid | Effervescent Disintegration System |
| Citric Acid | Effervescent Disintegration System |
| Hydroxypropyl Cellulose | Binder to facilitate roller compaction |
| Mannitol Granular | Diluent/Flavor enhancement |
| Xylitol | Sweetener/Flavor enhancement |
| FD&C Blue #1 (11-13%) Al Lake | Colorant |
| FD&C Yellow # 5 (15-18%) | Colorant |
| Al Lake | |
| FD&C Red #40 (14-17%) Al Lake | Colorant |
| Sucralose | Sweetener |
| Peppermint Flavor | Flavor |
| Spearmint Flavor | Flavor |
| Orange Flavor | Flavor |
| Poloxamer 188 | Surfactant, Dispersing |
| | Agent/Lubricant |

The invention is illustrated by the following Examples.

Each of the following three formulations are identical with the exception of colorants and flavors. Example I is peppermint flavored and contains a blue colorant, Example II is spearmint flavored and contains a combination of blue and yellow colorants to produce a mint green color, while Example III is orange flavored and contains red and yellow colorants to produce an orange color. It should be noted that 47.39 mg of CPC monohydrate is equivalent to 45 mg of CPC or 0.075% w/w CPC in 60 ml water.

### EXAMPLE I

TABLE 1
Composition of Example I

| | Roller Compaction | | |
|---|---|---|---|
| | EX17033 | | |
| Ingredient | mg/dose | %/dose | %/60 ml |
| Cetylpyridinium Chloride Monohydrate | 47.39 | 4.74 | 0.0790 |
| Menthol | 25.00 | 2.50 | 0.0417 |
| Sodium Bicarbonate #2 | 250.00 | 25.00 | 0.4167 |
| Tartaric Acid | 145.00 | 14.50 | 0.2417 |
| Citric Acid | 72.50 | 7.25 | 0.1208 |
| Hydroxypropyl Cellulose | 30.00 | 3.00 | 0.0500 |
| Mannitol Granular | 357.11 | 35.71 | 0.5952 |
| Xylitol | 15.00 | 1.50 | 0.0250 |
| FD&C Blue #1 (11-13%) Al Lake | 2.00 | 0.20 | 0.0033 |
| FD&C Yellow # 5 (15-18%) Al Lake | | | |
| FD&C Red #40 (14-17%) Al Lake | | | |
| Sucralose | 12.00 | 1.20 | 0.0200 |
| Peppermint Flavor | 20.00 | 2.00 | 0.0333 |
| Spearmint Flavor | | | |
| Orange Flavor | | | |
| Poloxamer 188 | 24.00 | 2.40 | 0.0400 |
| Totals: | 1000.00 | 100.00 | 1.67 |

### EXAMPLE II

TABLE 2
Composition of Example II

| | Roller Compaction | | |
|---|---|---|---|
| | EX17034 | | |
| Ingredient | mg/dose | %/dose | %/60 ml |
| Cetylpyridinium Chloride Monohydrate | 47.39 | 4.74 | 0.0790 |
| Menthol | 25.00 | 2.50 | 0.0417 |
| Sodium Bicarbonate #2 | 250.00 | 25.00 | 0.4167 |
| Tartaric Acid | 145.00 | 14.50 | 0.2417 |
| Citric Acid | 72.50 | 7.25 | 0.1208 |
| Hydroxypropyl Cellulose | 30.00 | 3.00 | 0.0500 |
| Mannitol Granular | 345.11 | 34.51 | 0.5752 |
| Xylitol | 15.00 | 1.50 | 0.0250 |
| FD&C Blue #1 (11-13%) Al Lake | 2.00 | 0.20 | 0.0033 |
| FD&C Yellow # 5 (15-18%) Al Lake | | | |
| FD&C Red #40 (14-17%) Al Lake | 2.00 | 0.20 | 0.0033 |
| Sucralose | 12.00 | 1.20 | 0.0200 |
| Peppermint Flavor | | | |
| Spearmint Flavor | 30.00 | 3.00 | 0.0500 |
| Orange Flavor | | | |
| Poloxamer 188 | 24.00 | 2.40 | 0.0400 |
| Totals: | 1000.00 | 100.00 | 1.67 |

### EXAMPLE III

TABLE 3
Composition of Example III

| | Roller Compaction | | |
|---|---|---|---|
| | EX17035 | | |
| Ingredient | mg/dose | %/dose | %/60 ml |
| Cetylpyridinium Chloride Monohydrate | 47.39 | 4.74 | 0.0790 |
| Menthol | 25.00 | 2.50 | 0.0417 |
| Sodium Bicarbonate #2 | 250.00 | 25.00 | 0.4167 |
| Tartaric Acid | 145.00 | 14.50 | 0.2417 |
| Citric Acid | 72.50 | 7.25 | 0.1208 |
| Hydroxypropyl Cellulose | 30.00 | 3.00 | 0.0500 |
| Mannitol Granular | 355.61 | 35.56 | 0.5927 |
| Xylitol | 15.00 | 1.50 | 0.0250 |
| FD&C Blue #1 (11-13%) Al Lake | | | |
| FD&C Yellow # 5 (15-18%) Al Lake | 3.00 | 0.30 | 0.0050 |
| FD&C Red #40 (14-17%) Al Lake | 0.50 | 0.05 | 0.0008 |
| Sucralose | 12.00 | 1.20 | 0.0200 |
| Peppermint Flavor | | | |
| Spearmint Flavor | | | |
| Orange Flavor | 20.00 | 2.00 | 0.0333 |
| Poloxamer 188 | 24.00 | 2.40 | 0.0400 |
| Totals: | 1000.00 | 100.00 | 1.67 |

### General Manufacturing Procedure

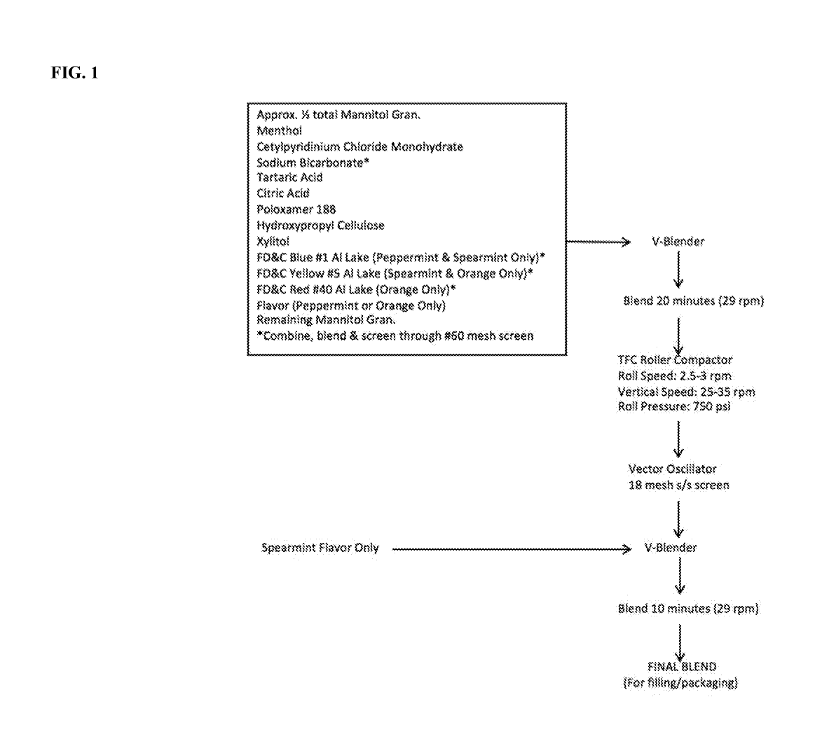

Examples I-III were prepared using the general manufacturing procedure shown in FIG. 1. Example II contained a microencapsulated spearmint flavor differing from the powdered peppermint and orange flavors used in Example I and Example III respectively. To avoid damaging the microencapsulation, the spearmint flavor was not included within the compaction but was added into the final blend. Otherwise the manufacturing process for all three trials is identical.

CPC was incorporated into the formulation at the monographed level of 0.075% w/w in 60 ml of water and menthol was incorporated at a level of 0.042% w/w. The physical characteristics of these three above examples were measured and are summarized in below.

### Physical Characteristics of Examples

The physical characteristics of all three examples are very similar in terms of material density and particle size distribution. The Carr's index is a calculation showing the percent difference between the tapped and bulk densities. Theoretically, values lower than 20% are indicative of very free-flowing materials and two of the examples trials indicate this. However from actual observations, all trials had very similar good flow characteristics. In addition, the made granules maybe non-hygroscopic and/or generally resistant to moisture uptake.

TABLE 4
Physical Characteristics

| Tests | Example I | Example II | Example III |
|---|---|---|---|
| Bulk Density: | 0.641 g/ml | 0.633 g/ml | 0.658 g/ml |
| Tapped Density: | 0.794 g/ml | 0.806 g/ml | 0.806 g/ml |
| Carr's Index: | 19.27% | 21.52% | 18.36% |
| US Mesh Size | % Retained | | |
| 20 | 13.8 | 10.1 | 10.1 |
| 30 | 22.1 | 23.8 | 23.8 |
| 40 | 14.6 | 16.2 | 15.1 |
| 60 | 15.8 | 17.2 | 20.8 |
| 80 | 6.8 | 7.8 | 11.0 |
| 100 | 8.5 | 9.6 | 12.7 |
| 120 | 6.9 | 6.2 | 3.5 |
| 200 | 9.4 | 7.6 | 3.0 |
| 230 | 1.7 | 1.2 | 0.3 |
| Pan | 0.4 | 0.3 | 0.0 |

Having thus described the invention in full detail above, it will be understood that such detail need not be strictly adhered to, but that additional changes and modifications may suggest themselves to one skilled in the art, all falling within the scope of the invention as defined by the subjoined claims.

1. A dry mouthwash granule for reconstitution in an aqueous medium, comprising:
an active component, an organic acid component and a carbonate salt component wherein said granule causes an effervescent reaction to occur upon being combined with said aqueous medium;
wherein said granule has a particle size between 53 microns and 1190 microns, and has a density of from about 0.5-1.2 g/cc; and
wherein said dry mouthwash granule is not a tablet.

2. The dry mouthwash granule as recited in claim 1, wherein said organic acid component comprises one or more acids selected from citric acid, malic acid, tartaric acid, ascorbic acid, fumaric acid, adipic acid, sodium hydrogen sulfate and mixtures thereof.

3. The dry mouthwash granule as recited in claim 2, wherein said organic acid comprises tartaric acid and citric acid.

4. The dry mouthwash granule as recited in claim 1, wherein said carbonate salt component is a carbonate salt selected from sodium carbonate, sodium bicarbonate, potassium carbonate, potassium bicarbonate, magnesium carbonate, calcium carbonate and mixtures thereof.

5. The dry mouthwash granule as recited in claim 4, wherein said carbonate salt is sodium bicarbonate.

6. The mouthwash granule as recited in claim 1, which substantially dissolves when placed in water of 60-150° F. in less than 30 seconds.

7. The dry mouthwash granule as recited in claim 1, wherein said granule has a particle size between 53 microns and 1000 microns.

8. The dry mouthwash granule as recited in claim 1, wherein said granule has a particle size between 74 microns and 1190 microns.

9. The dry mouthwash granule as recited in claim 1, further comprising a flavor component.

10. The dry mouthwash granule as recited in claim 9, wherein said flavor component is selected from peppermint flavor, spearmint flavor and orange flavor.

11. The dry mouthwash granule as recited in claim 1, wherein said active component is selected from an antimicrobial agent, remineralizing agent, anti-caries agent, anticalculus agent, moisturizing agent, breath freshening agent and desensitizing agents.

12. The dry mouthwash granule as recited in claim 11, wherein said active component is an antimicrobial agent.

13. The dry mouthwash granule as recited in claim 12, wherein said antimicrobial agent is cetylpyridinium chloride or a monohydrate thereof.

14. The dry mouthwash granule as recited in claim 1, further comprising a coloring agent.

15. The dry mouthwash granule as recited in claim 14, wherein said coloring agent is selected from FD&C Blue #1 Al Lake, FD&C Yellow #5 Al Lake, and FD&C Red #40 Al Lake.

16. The dry mouthwash granule as recited in claim 1, further comprising one or more sweetening agents.

17. The dry mouthwash granule as recited in claim 16, wherein said one or more sweetening agents are selected from mannitol, xylitol, and sucralose.

18. The dry mouthwash granule as recited in claim 1, further comprising a surfactant.

19. The dry mouthwash granule as recited in claim 18, wherein said surfactant is a poloxamer.

20. The dry mouthwash granule as recited in claim 19, wherein said poloxamer is poloxamer 188.

21. The dry mouthwash granule as recited in claim 1, further comprising a binder.

22. The dry mouthwash granule of claim 21, wherein said binder is hydroxypropyl cellulose.

23. The dry mouthwash granule of claim 1, which is non-hygroscopic.

24. A method of rinsing the mouth of a subject comprising the steps of:
a. combining the dry mouthwash granule of claim 1 with an aqueous medium in a container;
b. allowing the dry mouthwash granule to fully dissolve in said aqueous medium to form a solution; and
c. rinsing the mouth of said subject with said solution.
